(19) United States
(12) Patent Application Publication
(10) Pub. No.: US 2024/0164925 A1
(43) Pub. Date: May 23, 2024

(54) CATHETER FOR RAPID STENTING AT CORONARY ARTERY OSTIUM

(71) Applicant: IUCF-HYU ( INDUSTRY-UNIVERSITY COOPERATION FOUNDATION HANYANG UNIVERSITY ), Seoul (KR)

(72) Inventors: Young Hyo LIM, Seongnam-si (KR); Dae Sung KIM, Seoul (KR)

(73) Assignee: IUCF-HYU ( INDUSTRY-UNIVERSITY COOPERATION FOUNDATION HANYANG UNIVERSITY ), Seoul (KR)

(21) Appl. No.: 18/282,678

(22) PCT Filed: Mar. 14, 2022
(86) PCT No.: PCT/KR2022/003533

§ 371 Date: Sep. 18, 2023

(30) Foreign Application Priority Data
Mar. 19, 2021 (KR) 10-2021-0035827

(51) Int. Cl. A61F 2/958; A61F 2/954; A61M 25/09

(52) U.S. Cl. CPC A61F 2/958; A61F 2/954; A61M 25/09

(57) ABSTRACT

The present invention relates to a stent catheter for stenting, which enables a stent to be easily and quickly inserted into the coronary artery of the heart even in a state where the autonomic nerve causes the heart to beat 60 to 80 times per minute, a patient breathes at a heart rate of 12 to 20 beats per minute, and a patient uncooperatively moves, wherein a subtube having a predetermined length and allowing a support wire to pass therethrough is integrally coupled to the outer peripheral surface of a maintube through which a guide wire passes, such that an outlet of the subtube is located at the boundary of a balloon provided on the maintube. The present invention is designed to allow accurate and rapid insertion during the procedure at the ostium where the cardiac blood vessel begins or at the ostium where the branch blood vessel begins, so that rapid and easy stenting can be performed at the ostium of the coronary artery of the heart from the aorta through the insertion during the procedure for a patient showing emergent acute myocardial infarction caused by sudden blockage of the stable cardiac blood vessel, thereby enabling rapid response in case of emergency.

### TECHNICAL FIELD

The present invention relates to a stenting catheter, and more specifically to a stenting catheter that allows for easy and quick insertion of a stent into the ostium of a coronary artery of the heart or a branch vessel.

### BACKGROUND ART

In general, the heart receives oxygen and nutrients through three major blood vessels of the heart, called coronary arteries. Acute or chronic stenosis may occur in any one of the three coronary arteries due to causes such as arteriosclerosis, thrombosis, and vasoconstriction and spasm. Such stenosis is a reduction in the diameter of the passage of blood vessels or the like due to causes such as constriction. When this occurs, the supply of blood flow to the whole or part of the heart is reduced while the supply of oxygen and nutrients is rapidly reduced, causing the heart muscle to fall into a secondary ischemic state. This condition is called angina pectoris.

Angina pectoris is an ischemic heart disease and is classified as stable or unstable. Angina pectoris is a condition in which coronary arteries become considerably narrowed by atherosclerosis. Angina pectoris causes chest pain only when myocardial oxygen demand increases. It mainly appears during exercise and usually lasts for about 2-3 minutes, and chest pain disappears at rest. Another type of angina is variant angina, a condition in which ischemia occurs due to intermittent spasm of coronary arteries without structural changes of the blood vessels. Variant angina is characterized in that it usually occurs at dawn or in the morning and causes no pain in the daytime or during heavy exercise.

Unstable angina pectoris (USAP) is a heart disease that is classified as an acute coronary syndrome. In unstable angina pectoris, chest pain occurs even at rest, lasts longer and longer, and does not easily disappear despite the emergency use of nitroglycerin sublingual tablets. Unstable angina pectoris refers to a condition in which a coronary artery is completely blocked but which does not lead to myocardial necrosis. Another type of acute coronary syndrome is acute myocardial infarction (AMI), a condition in which a coronary artery is completely blocked by blood clots. Unlike unstable angina pectoris (USAP) in which myocardium is not yet necrotic, acute myocardial infarction (AMI) causes myocardial necrosis to release cardiac enzymes into the blood. Non-ST elevation MI (NSTEMI) is defined as acute myocardial infarction (AMI) without ST elevation. STEMI is defined as acute myocardial infarction (AMI) that is accompanied by ST elevation.

Acute myocardial infarction (AMI) is a disease that requires prompt emergency treatment. More than 90% of patients who received emergency treatment within 1 hour of onset of acute myocardial infarction return to normal but the survival rate drops below 50% after 8 hours of onset.

Balloon dilatation and stent crossover techniques are commonly used to treat narrowing of blood vessels by blood clots. These stenting techniques have many variables that are classified into emergency and non-emergency depending on how urgent they are. Stable angina and acute myocardial infarction (AMI) are typically classified into non-emergency and emergency, respectively.

FIG. 1 illustrates a form of stable angina without any branch vessel, in which blood vessels 1 are narrowed by blood clots 2. Specifically, when a blood vessel 1 is narrowed by blood clots 2, as illustrated in (a) of FIG. 2, a balloon 2 is dilated to break down the blood clots 2, as illustrated in (b) FIG. 2. A stent 4 is inserted between the broken blood clots (2), as illustrated in (d) of FIG. 2. The stent 4 is fixedly inserted into the blood vessel 1 narrowed by the blood clots 2 to widen the blood vessel 1 blocked by the blood clots 2, as illustrated in (e) of FIG. 2. This procedure is performed when relatively much time is available.

When acute myocardial infarction (AMI) occurs in the situation illustrated in FIG. 1, it can be treated in a relatively short time. The balloon is dilated before stenting.

A kissing balloon using two balloons is recommended for stable angina. FIG. 3 illustrates recommended locations of kissing balloons after stent placement. The kissing balloons can be located in various ways.

FIG. 4 illustrates stenting methods using kissing balloons. Referring to A, B, and D of FIG. 4, it is important to adapt stents to the ostia of blood vessels.

In many cases for acute myocardial infarction (AMI), major blood vessels are opened to stabilize the heart when side branch vessels are not blocked, and the branch vessels are opened during the secondary procedure. In some cases, the procedure may be given up if a therapeutic wire does not cross over.

For stable angina, there is time to make an effort to adapt stents to the ostia (OS) of blood vessels of the heart, as illustrated in FIGS. 5a and 5b. The heart beats 60 to 80 times per minute and the respiration rate is 12 to 20 breaths per minute. The heart rate is controlled by the autonomic nervous system. The position of the heart axis differs from patient to patient, making it difficult to take a tangential view during cine angiography. Further, it is difficult for patients to remain still for 30 minutes or longer. For these reasons, prompt and accurate treatment is necessary even for stable angina.

When it is intended to insert a stent into the ostium of the left anterior descending (LAD) artery or left circumflex (LCX) artery, a stent crossover technique is performed to insert the stent into the left anterior descending (LAD) artery or the left circumflex (LCX) artery from the left main coronary artery (LMCA). In contrast, when a stent crossover technique is performed at the aorta, it is necessary to accurately adapt the stent to the ostium of the left main coronary artery (LMCA) or right coronary artery (RCA), the ostium of the left anterior descending (LAD) artery or left circumflex (LCX) artery or the ostium of a branch vessel. However, this technique is difficult to perform because the heart is moving.

In conclusion, a severe degree of deviation of a stent from the ostium of a branch vessel, the left main coronary artery or the right coronary artery during stent placement is generally considered a mismatch but this is also a limitation of the stent crossover technique. Indeed, a stent is often placed beyond the ostium of a branch vessel, the left anterior descending artery or the left circumflex artery.

According to the prior art illustrated in FIG. 6, a stent S is inserted into the left main coronary artery (LMCA) or right coronary artery (RCA) from the aorta (Ao) of a patient's heart. In this case, if the stent protrudes from the ostium of the corresponding coronary artery, a catheter cannot be easily inserted into the ostium, making it difficult to inject a contrast medium into the corresponding coronary artery. As a result, the coronary artery cannot be accurately imaged using radioactive rays, making it impossible to accurately determine the location or degree of constriction. Moreover, when the procedure is forcibly carried out, a therapeutic guide wire should be passed through the flanks of the stent protruding from the ostium and a therapeutic balloon should be widened to pass the stent therethrough. Accordingly, in a situation in which urgent surgery is needed, the stent inserted into the ostium of the coronary artery acts as an obstacle that causes a waste of much time, putting the emergency patient at risk.

When it is intended to place a stent at the ostium of the left anterior descending (LAD) artery as a branch vessel of the heart, a stent inserted into the ostium of the left circumflex (LCX) artery acts as an obstacle that causes a waste of much time, putting the emergency patient at risk. Further, when it is intended to place a stent at the ostium of the left circumflex (LCX) artery as a branch vessel of the heart, a stent inserted into the ostium of the left anterior descending (LAD) artery acts as an obstacle that causes a waste of much time, putting the emergency patient at risk.

In the worst case during stenting, an already inserted stent tends to be damaged, causing the problem that an additional procedure or surgery should be performed to take the damaged stent out of the heart.

FIG. 7 illustrates a catheter 10 for the examination and treatment of a blood vessel of the heart in which a hub 11 is coupled to one end of a tube a tip 12 is formed at the other end of the tube. A wire is inserted into the hub 11. The tip portion of the catheter 10 is curved at a predetermined angle to form a first curve 13, further curved at a predetermined angle to form a second curve 14 that is spaced a distance from the first curve 13, and further curved at a predetermined angle to form a third curve 15 that is spaced a distance from the second curve 14. The catheter 10 has a length of approximately 100 to 110 cm.

Various types of catheters or guide wires for the treatment of blood vessels of the heart usually use patients' right wrists or they use patients' left wrists or femoral arteries depending on the patients' conditions. Approximately 7 or 10 cm long catheter sheaths are used to insert the catheters or guide wires.

Catheters of various shapes are used for right and left blood vessels of the heart. Specifically, first to third curves of approximately 250 or more shapes are provided depending on the positions of catheters inserted, for example, the ostia of right and left blood vessels of the heart and the wrists or thighs.
(a) of FIG. 8 illustrates a conventional over-the-wire balloon catheter 20. The catheter includes a tube 21 of a predetermined length, a radially inflating balloon 25 provided at one side of the tube 21, a tip 26 which is provided at the right end of the balloon 25 and through which a guide wire 24 penetrating the tube 21 is exposed to the outside, a hub 22 which is provided at the left end of the tube and into which the wire 24 is inserted, and an inflation port 23 through which a liquid is injected into the balloon 25 and which is connected to the hub in parallel. (b) of FIG. 8 illustrates a monorail balloon catheter 20 having a structure in which a wire 24 is inserted into one side of a tube 21.
(a) of FIG. 9 illustrates a conventional stent catheter 30 having a structure in which a radially inflating balloon 33 is provided at one end of a tube 31 where a tip 32 is formed and a stent 34 is attached to the outer circumference of the balloon 33. In this structure, a distance x is created between the right end of the tube 31 connected to the balloon 33 and the left end of the stent 34 located on the outer circumference of the balloon 33. The stent 34 expands when the balloon 33 is inflated, as illustrated (b) of FIG. 9. The right and left ends of the stent 34 are equidistant from the surface center of the balloon 33.

FIG. 10 exemplifies the use of a conventional stent catheter 30 for stenting when blood clots 2 are formed at the ostium of a blood vessel of the heart, for example, at the ostium of the left main coronary artery (LMCA) as a blood vessel 1 connected to the aorta of the heart. In an unstable situation in which a patient's heart is beating and the patient is breathing, a catheter 10 for examination and treatment and the stent catheter 30 cannot be placed at desired locations. For this reason, unlike in FIG. 10b in which the stent 34 is accurately placed at the ostium of a blood vessel, the stent 34 is placed protruding outward from the ostium of the blood vessel 1, as illustrated in FIG. 10a, or the stent 34 is inserted inward from the ostium of the blood vessel 1, as illustrated in FIG. 10c, with the result that the stent 34 cannot be accurately placed at an accurate location on the blood clots 2 formed at the ostium of the blood vessel 1 because forces moving from side to side due to heartbeat and breathing act on the stent.

FIG. 11 illustrates the use of a conventional stent catheter 30 for stenting when blood clots 2 are formed at the ostium of a branch vessel of the heart, for example, at the ostium of the left anterior descending (LAD) artery as a branch 1 from the left main coronary artery (LMCA). In an unstable situation in which a patient's heart is beating and the patient is breathing, a catheter 10 for examination and treatment and the stent catheter 30 cannot be placed at desired locations. For this reason, unlike in FIG. 11b in which the stent 34 is accurately placed at the ostium of a blood vessel, the stent 34 is placed protruding outward from the ostium of the left anterior descending (LAD) artery, as illustrated in FIG. 11a, or the stent 34 is inserted inward from the ostium of the left anterior descending (LAD) artery, as illustrated in FIG. 11c, with the result that the stent 34 cannot be accurately placed at an accurate location on the blood clots 2 formed at the ostium of the left anterior descending (LAD) artery because forces moving from side to side due to heartbeat and breathing act on the stent.

### DETAILED DESCRIPTION OF THE INVENTION

### Problems to be Solved by the Invention

The present invention has been made in an effort to solve the above-described problems, and one object of the present invention is to provide a stenting catheter that allows for easy and quick insertion of a stent into the ostium of a coronary artery of the heart or a branch vessel. Another object of the present invention is to minimize obstacles during a restenting procedure for stable angina or emergent acute myocardial infarction.

### Means for Solving the Problems

An aspect of the present invention provides a catheter for rapid stenting at the ostium of a blood vessel of the heart, including a main tube through which a guide wire passes and a sub-tube of a predetermined length through which a support wire passes and which is integrally attached to the outer circumference of the main tube wherein the sub-tube has an outlet coupled to a balloon provided on the main tube at the boundary with the balloon.

A stent may be attached to the outer circumference of the balloon.

One end of the stent attached to the outer circumference of the balloon may be adapted and coupled to the outlet of the sub-tube.

The guide wire may be introduced into a blood vessel of the heart or a branch vessel with blood clots and the support wire may be supported in contact with the outer wall of the ostium of the blood vessel of the heart or the inner wall of the ostium of another one other than the branch vessel.

### Effects of the Invention

The catheter of the present invention can be accurately and quickly inserted into the ostium of a blood vessel of the heart or a branch vessel. As a result, the catheter of the present invention can be quickly and easily inserted into the ostium of a coronary artery of the heart from the aorta during treatment of a patient with emergent acute myocardial infarction in which a stable blood vessel of the heart is suddenly blocked.

### BRIEF DESCRIPTION OF THE DRAWINGS

FIG. 1 schematically illustrates a state in which blood vessels of the heart without branch vessels are narrowed by blood clots.

FIG. 2 schematically and diagrammatically illustrates a stenting procedure after balloon dilatation in a blood vessel of the heart without any branch vessel.

FIG. 3 schematically illustrates recommended locations of kissing balloons in branch vessels.

FIG. 4 schematically and diagrammatically illustrates various exemplary procedures for kissing balloon dilatation.

FIG. 5a schematically illustrates blood clots formed at the ostia of blood vessels of the heart.

FIG. 5b schematically illustrates the ostia of blood vessels of the heart and branch vessels.

FIG. 6 schematically illustrates incorrect stenting procedures at the ostia of coronary arteries from the aorta.

FIG. 7 schematically illustrates (a, b) a catheter for the examination or treatment of a blood vessel of the heart.

FIG. 8 schematically illustrates (a, b) conventional balloon catheters.

FIG. 9 schematically illustrates (a, b) major parts of a conventional balloon catheter.

FIGS. 10a to 10c schematically illustrate exemplary stenting procedures using conventional balloon catheters at the ostia of blood vessels of the heart.

FIGS. 11a to 11c schematically illustrate exemplary stenting procedures using conventional balloon catheters at the ostia of branch vessels.

FIG. 12 schematically illustrates (a, b) balloon catheters for rapid stenting at the ostia of blood vessels of the heart according to exemplary embodiments of the present invention.

FIG. 13 schematically illustrates (a, b) major parts of a balloon catheter for rapid stenting at the ostium of a blood vessel of the heart according to the present invention.

FIGS. 14a to 14g schematically illustrate an exemplary procedure for rapid stenting using a balloon catheter of the present invention at the ostium of a blood vessel of the heart.

FIGS. 15a to 15g schematically illustrate an exemplary procedure for rapid stenting using a balloon catheter of the present invention at the ostium of a branch vessel.

FIG. 16 schematically illustrates the examination and treatment of blood vessels of the heart using a catheter by hanging the catheter on the right and left ostia of the heart.

FIG. 17 schematically illustrates the distribution of a force in right, left, upward, and downward directions using (a) a single wire or (b) two wires from a catheter for examination and treatment despite shaking of the heart.

FIG. 18 schematically illustrates the distribution of a force in right, left, upward, and downward directions using (a) a single wire or (b, c) two wires from a catheter for examination and treatment to introduce the wire(s) into a blood vessel of the heart and a branch vessel despite shaking of the heart.

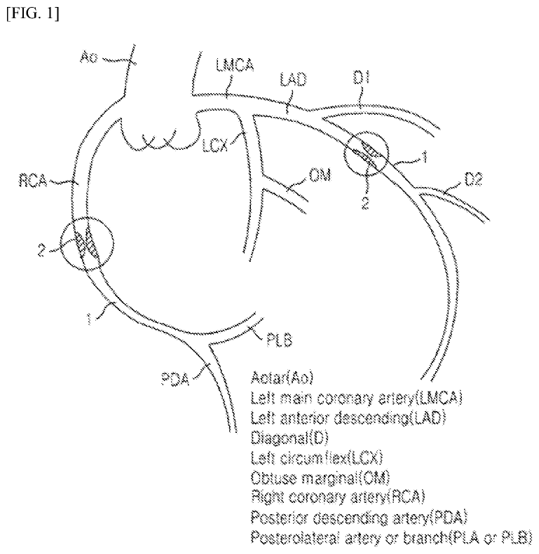

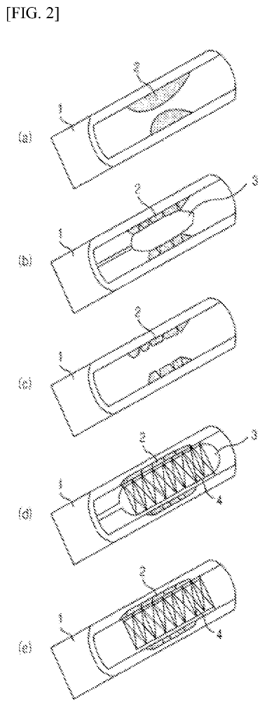

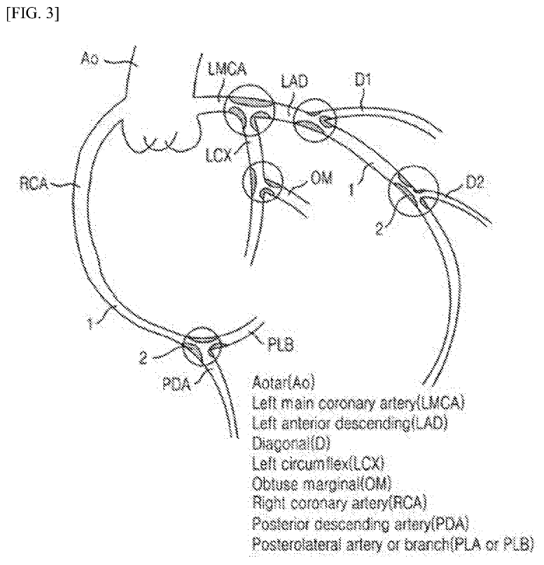

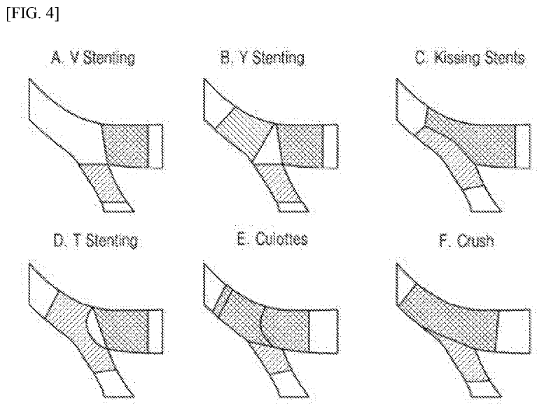

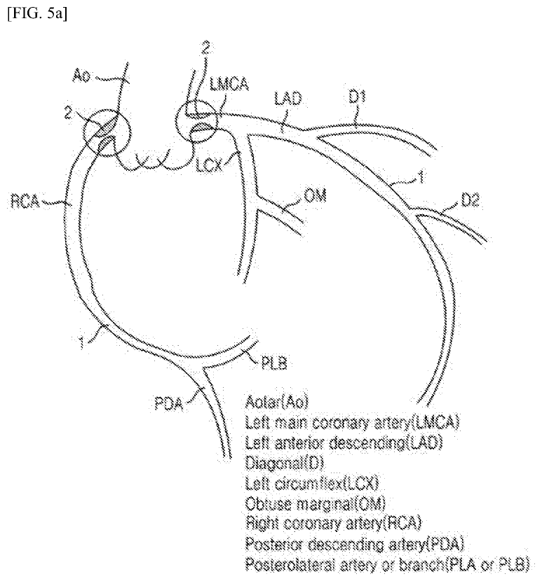

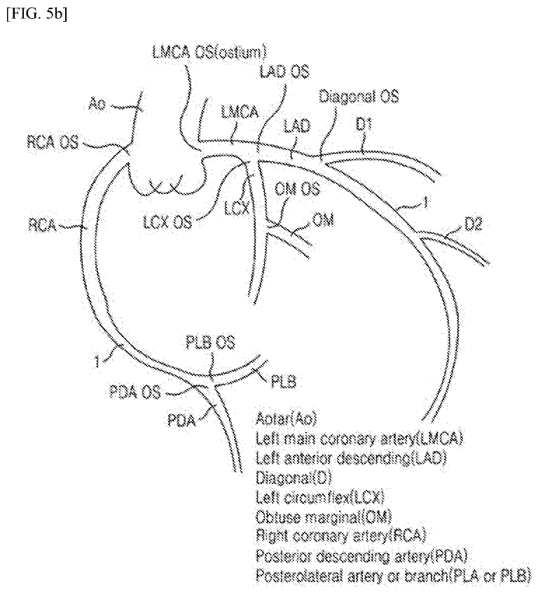

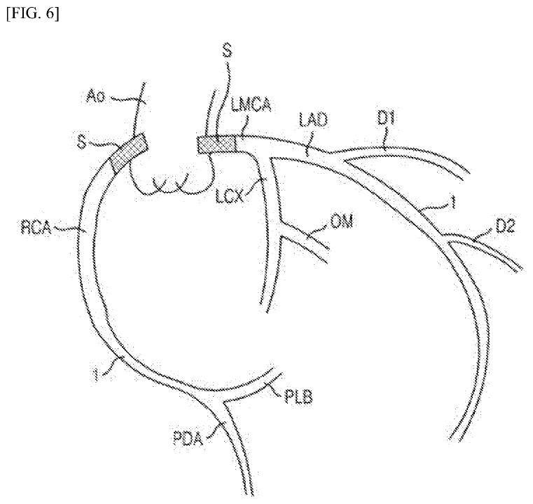

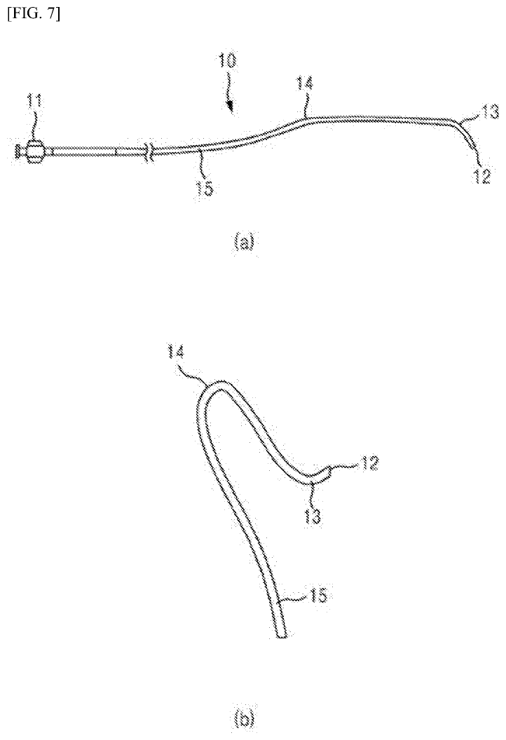

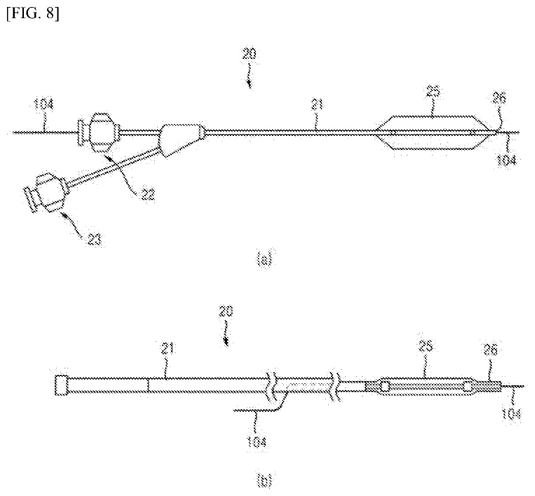

### BEST MODE FOR CARRYING OUT THE INVENTION

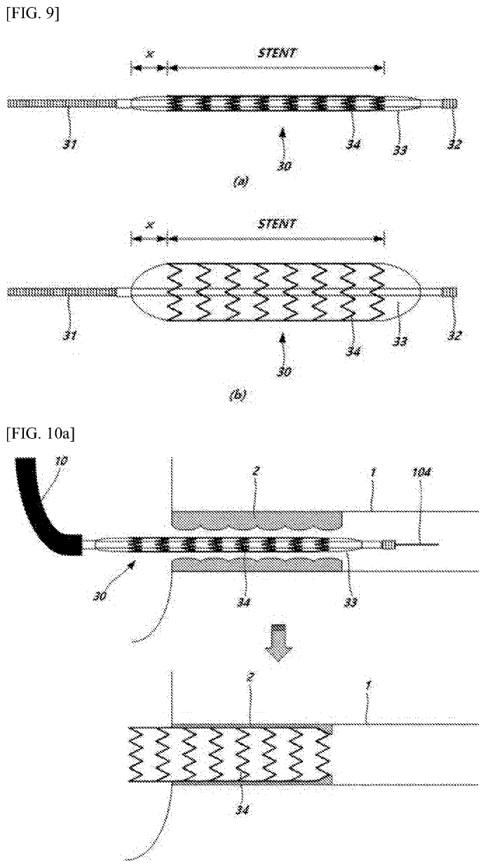

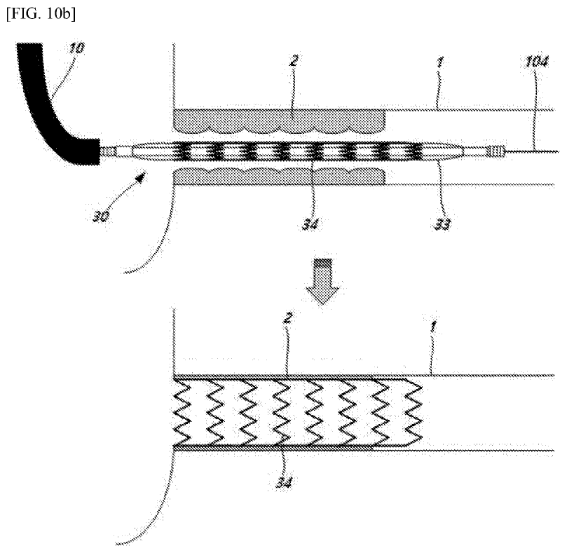

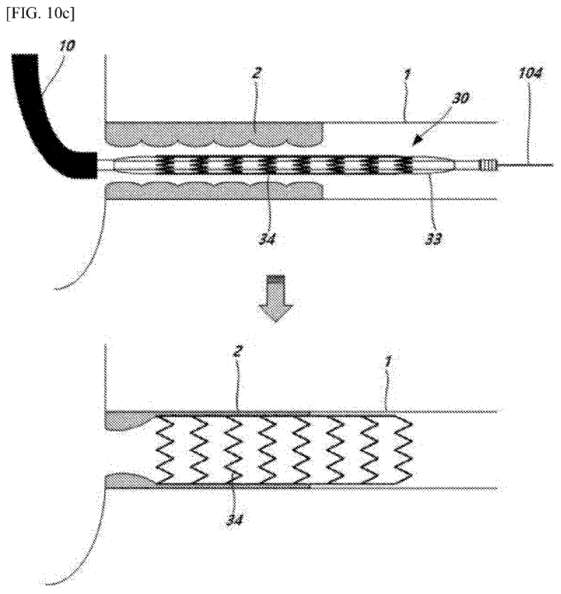

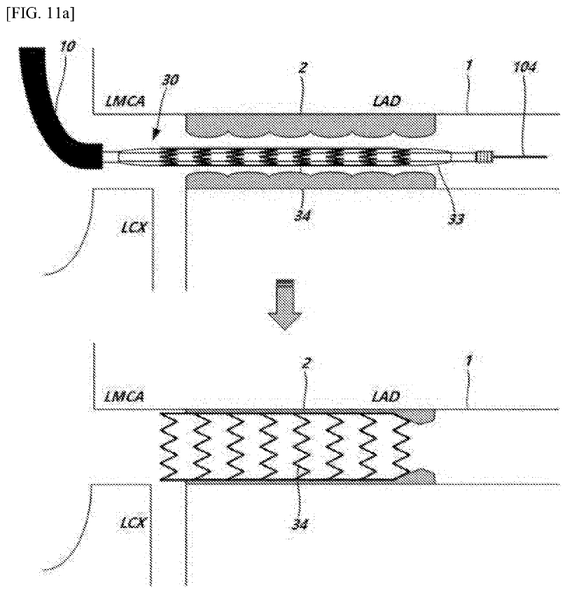

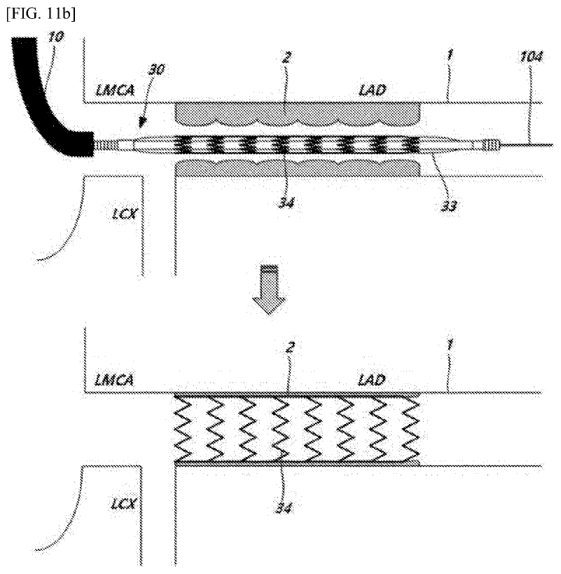

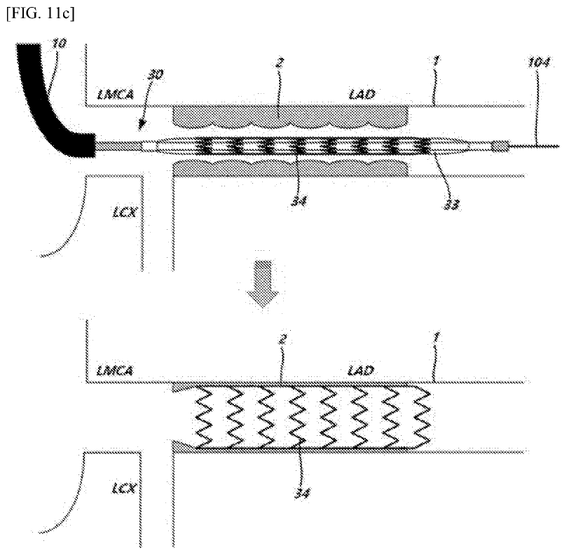

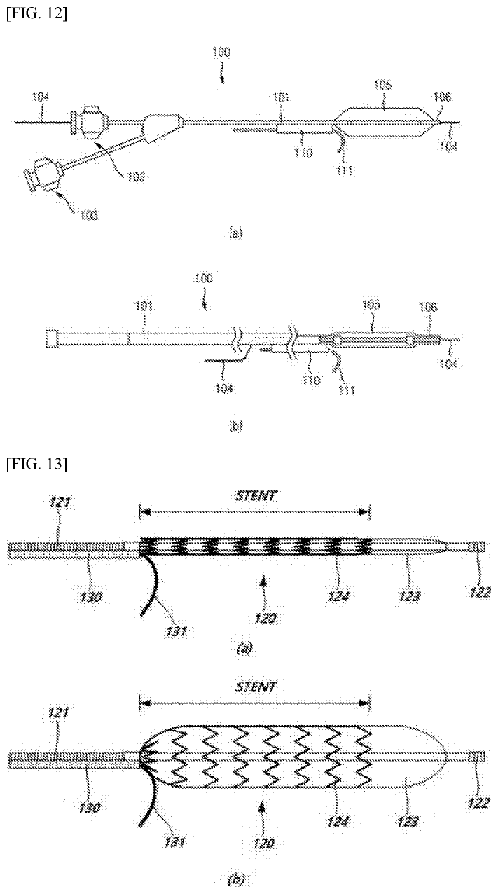

Now, exemplary embodiments of a catheter for rapid stenting at the ostium of a blood vessel of the heart according to the present invention will be described in detail with reference to the accompanying drawings.
(a) of FIG. 12 illustrates a balloon catheter with a guide wire (over-the-wire balloon catheter) 100. The balloon catheter includes a main tube 101 having a predetermined length, a balloon 105 coupled to the right end of the main tube 101 and inflated radially by a liquid injected from the outside, a guide wire 104 provided at the right end of the balloon 105 and penetrating the main tube 101, a tip 106 through which the guide wire is exposed to the outside, a hub 102 which is coupled to the left side of the main tube 101 and into which the guide wire 104 is inserted, and an inflation port 103 connected to an indeflator for injecting the liquid into the balloon 105 and provided in parallel with the hub 102. In the balloon catheter 100 of the present invention, a sub-tube 110 is integrally attached to the outer circumference of the main tube 101. The sub-tube 110 has a predetermined length and its right end extends and is coupled to the left end of the dilated balloon 105. That is, the right end of the sub-tube 110 extends to the boundary with the balloon 105 and is coupled to the left end of the balloon 105. A support wire 111 is inserted into the sub-tube 110 from the outside of the human body. The support wire 111 is supported in contact with the wall of the ostium of a blood vessel in a vertical, horizontal or diagonal direction after being drawn out from the sub-tube 110. When the balloon catheter 100 is inflated to push blood clots out of main portions of a blood vessel, for example, the ostium of a blood vessel, shaking of the catheter in a certain direction due to patient's heartbeat or breathing is minimized, enabling inflation of the balloon in its correct position.
(b) of FIG. 12 illustrates the structure of a monorail balloon catheter 100 in which a guide wire 104 is inserted into one side of a main tube 101. Also in this structure, a sub-tube 110 into which a support wire 111 is inserted and from which the support wire 111 is drawn out is integrally provided with the main tube 101.
(a) of FIG. 13 illustrates a stent catheter 120 having a structure in which a radially inflating balloon 123 is provided at the right end of a main tube 121, a tip 122 through which a guide wire is exposed is formed at the right side of the balloon 123, and a stent 124 is attached to the outer circumference of the balloon 123. The gap between the right end of the main tube 121 connected to the balloon 123 and the left end of the stent 124 positioned on the outer circumference of the balloon 123 is close to approximately zero (0) (see “x” in FIG. 9).

In the stent catheter 120 of the present invention, a sub-tube 130 is integrally attached to the outer circumference of the main tube 121. The sub-tube 130 has a predetermined length and its right end extends and is coupled to the left end of the dilated balloon 105. A support wire 131 is inserted into the sub-tube 130 from the outside of the human body. The support wire 131 is supported in contact with the wall of the ostium of a blood vessel in a vertical, horizontal or diagonal direction after being drawn out from the sub-tube 130. When the balloon catheter 120 is inflated to push blood clots out of main portions of a blood vessel, for example, the ostium of a blood vessel, shaking of the catheter in a certain direction due to patient's heartbeat or breathing is minimized, enabling inflation of the balloon in its correct position to fix the stent 124 to the wall of the blood vessel. The inflation of the balloon 123 of the stent catheter 120 leads to expansion of the stent 124 coupled to the surface of the balloon 123, as illustrated in (b) of FIG. 13.

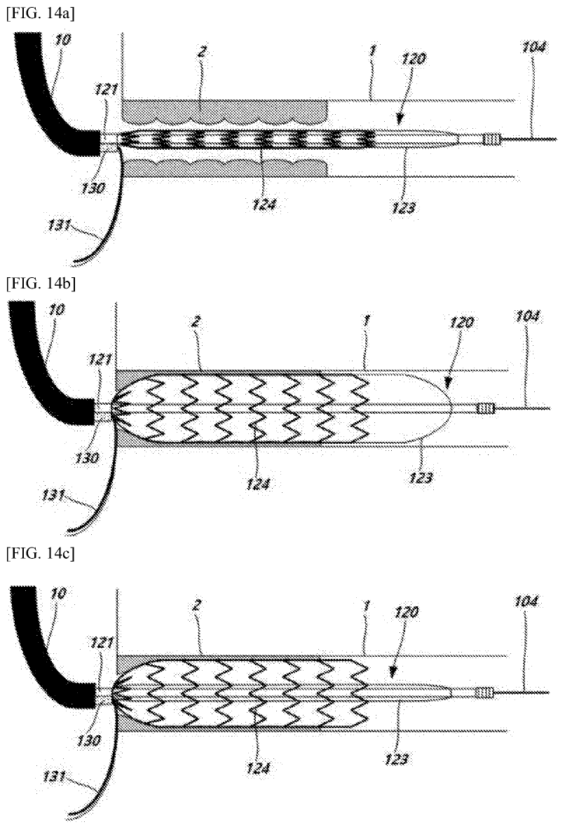

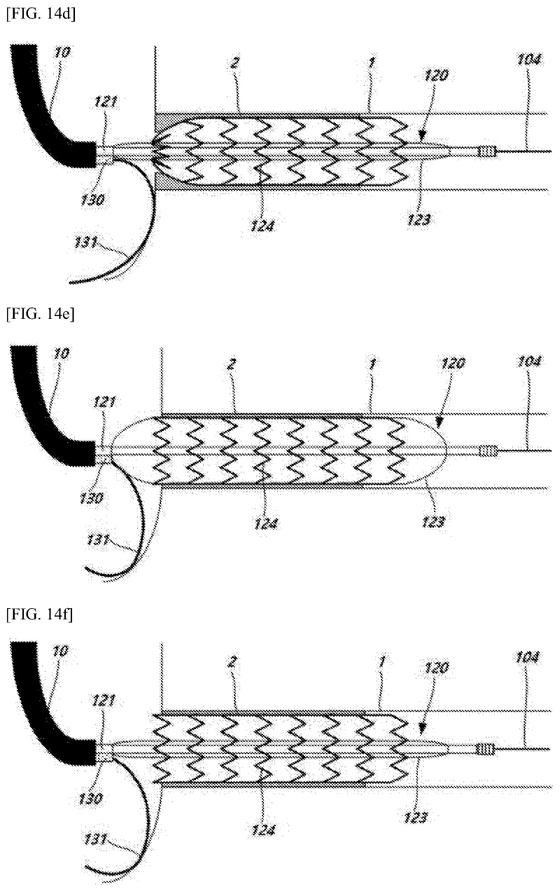

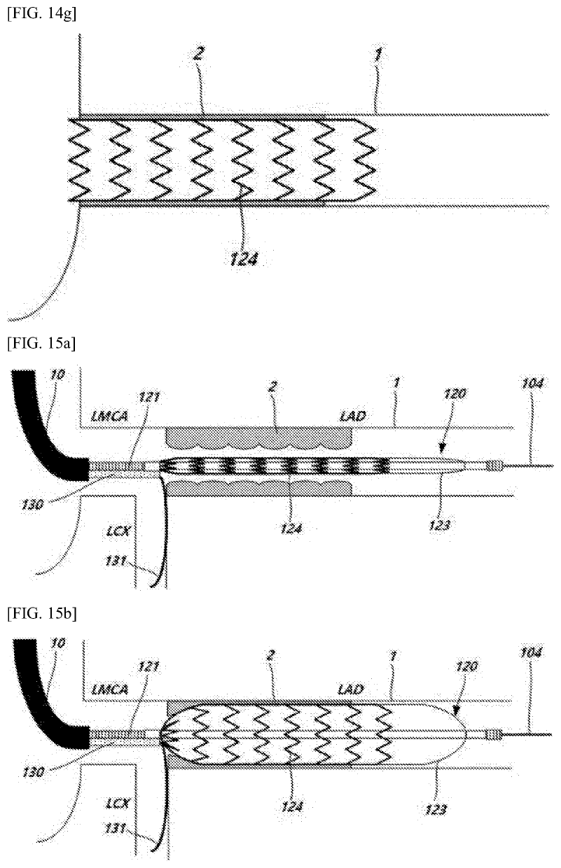

FIGS. 14a to 14g schematically illustrate a procedure for placing the stent 124 at the ostium of a blood vessel of the heart using the stent catheter 120, particularly for placing the stent 124 on blood clots 2 formed at the ostium of the left main coronary artery (LMCA) or right coronary artery (RCA) as a blood vessel of the heart.

FIG. 14a illustrates a state in which a tip of the stent catheter 120 is inserted into a blood vessel 1 of the heart through a catheter 10. The support wire 131 of a predetermined length exposed through the sub-tube 130 of the stent catheter 120 is brought into contact with the side of the ostium of the blood vessel 1. Accordingly, in a state in which a tip of the catheter 10 is positioned at the ostium of the blood vessel 1, the stent catheter 120 is inserted into the blood vessel 1 in an approximately horizontal direction and the support wire 131 exposed through the sub-tube 130 comes into contact with the wall of the blood vessel around the ostium of the blood vessel 1 in an approximately vertical direction. As a result, when a patient's heart is shaken right, left, up and down due to the patient's heartbeat or breathing, the tip of the stent catheter 120 is also shaken in the same directions so that the stent catheter 120 can be more rapidly and stably placed at the ostium of the blood vessel 1.

Referring to FIG. 14b, when the tip of the stent catheter 120 is placed at a desired location inside the ostium of the blood vessel 1, the balloon 123 provided in the stent catheter 120 is inflated and the stent 124 coupled to the surface of the balloon 123 expands and is fixed to the surface of the blood clots 2. Also at this time, the support wire 131 penetrating the sub-tube 130 comes into contact with the wall of the blood vessel around the ostium of the blood vessel 1 to support the stent catheter 120.

Referring to FIG. 14c, the inflated balloon 123 is deflated to separate the stent 124 from the balloon 123. When the balloon 123 is deflated, the left end of the stent 124 does not fully expand by the inflated shape of the left edge of the balloon 123 inclined at a certain angle.

Thus, the balloon 123 retracts the tip of the contracted stent catheter 120 by a predetermined distance from the ostium of the blood vessel 1, so that the left end of the stent 124 is positioned on the surface of the balloon 123 where the balloon 123 is maximally dilated (see FIG. 14d). Referring then to FIG. 14e, the balloon 123 is again inflated through the stent catheter 120. When the balloon 123 is inflated, the left end of the stent 124 fully expands and is fixed to the surface of the blood clots 2. Also at this time, the support wire 131 penetrating the sub-tube 130 comes into contact with the wall of the blood vessel around the ostium of the blood vessel 1 to support the stent catheter 120.

Referring to FIG. 14f, after the stent 124 fully expands and is fixed to the blood clots 2, the balloon 123 is deflated. Referring then to FIG. 14g, both the stenting catheter 120 and the catheter 10 are withdrawn from the blood vessel 1 and the aorta Ao, completing the stenting procedure.

### MODE FOR CARRYING OUT THE INVENTION

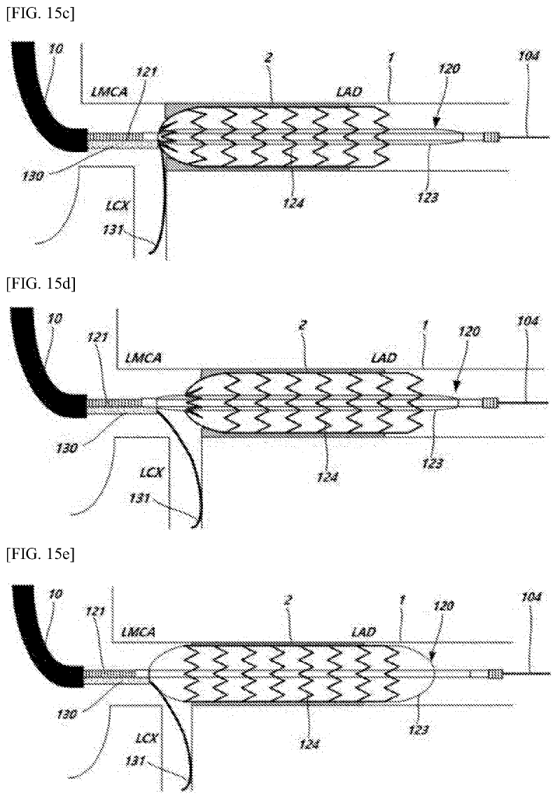

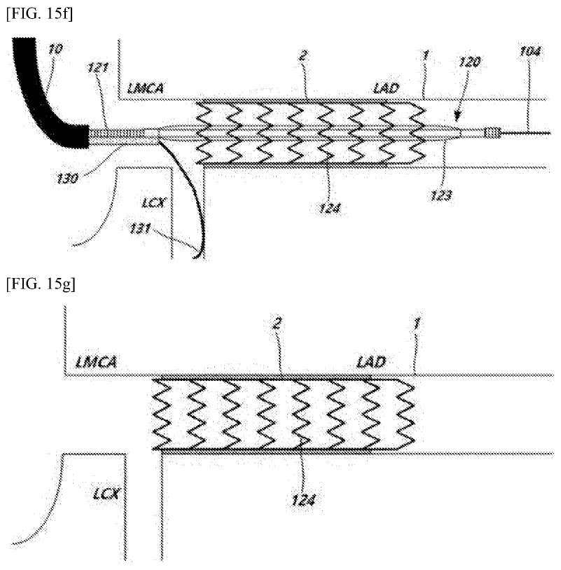

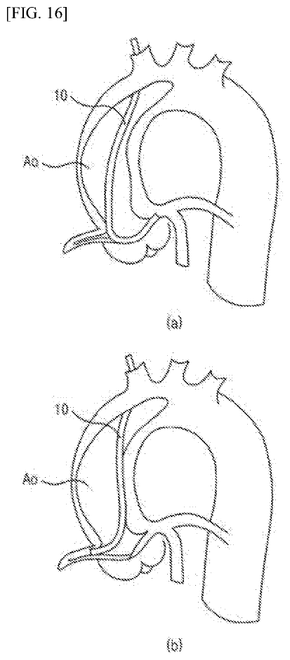

Next, FIGS. 15a to 15g schematically illustrate a procedure for placing the stent 124 at the ostium of a blood vessel of the heart using the stent catheter 120, particularly for placing the stent 124 on blood clots 2 formed at the ostium of a branch vessel from the left main coronary artery (LMCA) or right coronary artery (RCA) as a blood vessel of the heart, for example, at the ostium of the left anterior descending (LAD) artery or left circumflex (LCX) artery from the left main coronary artery (LMCA) or at the ostium of the posterior descending artery (PDA) or posterolateral branch (PLB) from the right coronary artery (RCA).

FIG. 15a illustrates a state in which a tip of the stent catheter 120 is inserted into a branch vessel 1 of the heart, that is, the left anterior descending (LAD) artery as a branch from the left main coronary artery (LMCA) when blood clots 2 are formed at the ostium of the left anterior descending (LAD) artery, through the catheter 10. The support wire 131 of a predetermined length exposed through the sub-tube 130 of the stent catheter 120 is brought into contact with the inner wall of the ostium of the left circumflex (LCX) artery as a branch from the left main coronary artery (LMCA). Accordingly, in a state in which a tip of the catheter 10 is positioned inside the ostium of the left main coronary artery (LMCA), the tip of the stent catheter 120 is inserted into the ostium of the left main coronary artery (LMCA) in an approximately horizontal direction and the support wire 131 exposed through the sub-tube 130 comes into contact with the inner wall of the ostium of the left circumflex (LCX) artery in an approximately vertical direction. As a result, when a patient's heart is shaken right, left, up and down due to the patient's heartbeat or breathing, the tip of the stent catheter 120 is also shaken in the same directions so that the stent catheter 120 can be more rapidly and stably placed at the ostium of the blood vessel 1.

Referring to FIG. 15b, when the tip of the stent catheter 120 is placed at a desired location inside the ostium of the left anterior descending (LAD) artery as a branch vessel, the balloon 123 provided in the stent catheter 120 is inflated and the stent 124 coupled to the surface of the balloon 123 expands and is fixed to the surface of the blood clots 2. Also at this time, the support wire 131 penetrating the sub-tube 130 comes into contact with the inner wall of the ostium of the left circumflex (LCX) artery as a branch vessel to support the stent catheter 120.

Referring to FIG. 15c, the inflated balloon 123 is deflated to separate the stent 124 from the balloon 123. When the balloon 123 is deflated, the left end of the stent 124 does not fully expand by the inflated shape of the left edge of the balloon 123 inclined at a certain angle.

Thus, the balloon 123 retracts the tip of the contracted stent catheter 120 by a predetermined distance from the ostium of the left anterior descending (LAD) artery as a branch vessel, so that the left end of the stent 124 is positioned on the surface of the balloon 123 where the balloon 123 is maximally dilated (see FIG. 15d). Referring then to FIG. 15e, the balloon 123 is again inflated through the stent catheter 120. When the balloon 123 is inflated, the left end of the stent 124 fully expands and is fixed to the surface of the blood clots 2. Also at this time, the support wire 131 penetrating the sub-tube 130 comes into contact with the inner wall of the ostium of the left circumflex (LCX) artery as a branch vessel to continuously support the stent catheter 120.

Referring to FIG. 15f, after the stent 124 fully expands and is fixed to the blood clots 2, the balloon 123 is deflated. Referring then to FIG. 15g, both the stenting catheter 120 and the catheter 10 are withdrawn from the branch vessel and the aorta Ao, completing the stenting procedure.

As described above, the catheter of the present invention is designed for rapid stenting at the ostium of a blood vessel of the heart and is constructed as a stent or balloon catheter having a structure in which a sub-tube is integrally provided with a main tube and a support wire is inserted into and penetrates the sub-tube. This structure enables fast and stable stenting despite shaking of the heart due to patient's heartbeat or breathing when the balloon catheter is used to inflate a balloon or the stent catheter is used to inflate a balloon and expand and fix a stent.

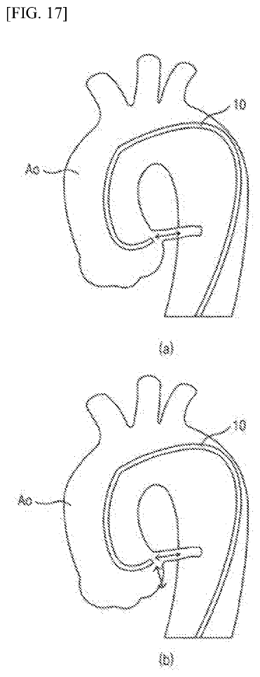

The guide wire and the catheter for examination and treatment are inserted into the aorta through an artery of the wrist or a femoral artery of the leg depending on the patient's conditions. As illustrated in (a) of FIG. 16, the tips of the guide wire and the catheter for examination and treatment inserted into the aorta (Ao) are placed at the ostium of the left main coronary artery (LMCA), followed by examination or treatment. At this time, a contrast medium is injected or the balloon or stent catheter is inserted through the catheter for examination and treatment. As illustrated in (b) of FIG. 16, the tip of the catheter for examination and treatment is placed at the ostium of the right coronary artery (RCA), followed by examination or treatment.

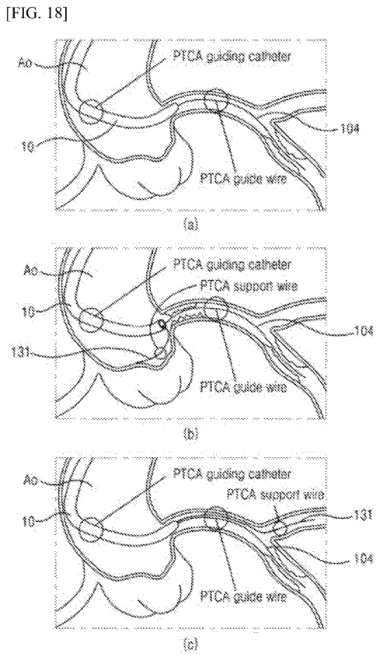

In the case where a single guide wire is inserted from the catheter for examination and treatment, as illustrated in (a) of FIG. 17, shaking occurs in the right and left directions at the ostium of the blood vessel of the heart due to patient's heartbeat or breathing, making it difficult to perform balloon dilatation or stent placement. In contrast, in the case where the guide wire and the support wire are inserted from the catheter for examination and treatment, as illustrated in (b) of FIG. 17, the support wire is fixed in contact with the ostium or wall of a blood vessel of the heart or a branch vessel despite shaking of the heart due to patient's heartbeat or breathing, ensuring fast and stable introduction of the guide wire into the blood vessel where blood clots are located.
(a) of FIG. 18 illustrates a normal case in which only one guide wire is used in the catheter for examination and treatment. In this case, the guide wire is not easy to insert into a place where blood clots are located along a blood vessel of the heart or a branch vessel. In contrast, in a state in which the catheter for examination and treatment is positioned at the ostium of a blood vessel of the heart, as illustrated in (b) of FIG. 18, the guide wire drawn from the catheter for examination and treatment is inserted into a place where blood clots are located along the blood vessel of the heart and the support wire is brought into contact with the outer wall of the ostium of the blood vessel of the heart, enabling fast and stable treatment at the ostium of the blood vessel of the heart. Alternatively, in a state in which the catheter for examination and treatment is positioned at the ostium of a blood vessel of the heart, as illustrated in (c) of FIG. 18, the guide wire drawn from the catheter for examination and treatment is inserted into a place where blood clots are located in a branch vessel from the blood vessel of the heart and the support wire is inserted into and fixedly support another one other than the branch vessel, enabling fast and stable treatment at the ostium of the branch vessel.

### EXPLANATION OF REFERENCE NUMERALS

Ao: Aorta, 1: Blood vessel, 2: Blood clot, 3: Balloon, 4, S: Stent, 10: Catheter for examination and treatment, 11: Hub, 12: Tip, 13: First curve, 14: Second curve, 15: Third curve, 20: Balloon catheter, 21: Tube, 22: Hub, 23: Inflation port, 24: Wire, 25: Balloon, 26: Tip, 30: Stent catheter, 31: Tube, 32: Tip, 33: Balloon, 34: Stent, 100: Balloon catheter, 101: Main tube, 102: Hub, 103: Inflation port, 104: Guide wire, 105: Balloon, 106: Tip, 110: Sub-tube, 111: Support wire, 120: Stent catheter, 121: Main tube, 122: Tip, 123: balloon, 124: Stent, 130: Sub-tube, 131: Support wire

### INDUSTRIAL APPLICABILITY

The stenting catheter of the present invention allows for quick and easy insertion of a stent into the ostium of a coronary artery of the heart from the aorta during treatment of a patient with emergent acute myocardial infarction. Therefore, the catheter of the present invention is suitable for use in treating emergent acute myocardial infarction.

1. A catheter for rapid stenting at the ostium of a blood vessel of the heart, comprising a main tube through which a guide wire passes and a sub-tube of a predetermined length through which a support wire passes and which is integrally attached to the outer circumference of the main tube wherein the sub-tube has an outlet coupled to a balloon provided on the main tube at the boundary with the balloon.

2. The catheter according to claim 1, wherein a stent is attached to the outer circumference of the balloon.

3. The catheter according to claim 2, wherein one end of the stent attached to the outer circumference of the balloon is adapted and coupled to the outlet of the sub-tube.

4. The catheter according to claim 1, wherein the guide wire is introduced into a blood vessel of the heart or a branch vessel with blood clots and the support wire is supported in contact with the outer wall of the ostium of the blood vessel of the heart or the inner wall of the ostium of another one other than the branch vessel.
